(19) United States
(12) Patent Application Publication
(10) Pub. No.: US 2020/0254103 A1
(43) Pub. Date: Aug. 13, 2020

(54) Method for treating neurological conditions and improving human cognition

(71) Applicants: John Mee, Stuart, FL (US); Dean Radin, Petaluma, CA (US)

(72) Inventors: John Mee, Stuart, FL (US); Dean Radin, Petaluma, CA (US)

(21) Appl. No.: 16/272,138

(22) Filed: Feb. 11, 2019

(51) Int. Cl. A61K 47/50; A61K 38/46; C12N 15/113; A61P 25/28; A61K 9/00

(52) U.S. Cl. CPC A61K 47/50; A61K 38/465; A61K 9/0009; A61P 25/28; C12N 15/113

(57) ABSTRACT

A method of treating neurological conditions and improving human cognition by modifying neurons in targeted brain regions, comprising administering a neuron editing biologic in combination with a brain region activator which directs the biologic to a specific area in the brain.

### FIELD OF THE INVENTION

The present invention relates generally to human genetic engineering, and more particularly to the application of methods and techniques for delivering CRISPR to specific targeted regions in the adult human brain.

### BACKGROUND OF THE INVENTION

The present invention generally relates to a method for delivering CRISPR biologics to distinct areas of the brain. Current CRISPR vectoring methods cannot distinguish between brain regions; hence, CRISPR cannot presently be used to explicitly treat conditions which affect or arise from specific areas of the brain, or to enhance cognitive abilities which involve neurons in particular regions of the brain.

### SUMMARY OF THE INVENTION

The present invention provides a method of treating neurological conditions and improving human cognition by modifying neurons in targeted brain regions, comprising administering a neuron editing biologic in combination with a brain region activator which directs the biologic to a specific area(s) in the brain.

It is a principle object of the present invention to provide a new technology for delivering CRISPR biologics to any targeted brain region.

It is a specific object of the invention to enable new types of CRISPR applications which target specific brain regions.

It is a further object of the invention to provide a flexible, general-purpose CRISPR delivery system which can be customized to meet individual application needs.

It is a another object of the invention to facilitate new CRISPR applications which treat neurological conditions affecting specific brain regions such as ADD/ADHD, traumatic memory/PTSD, OCD, stress disorders, anxiety, depression, sleep issues, memory concerns, concussions, Tourette Syndrome, psychosomatic issues and tinnitus.

It is a further object of the invention to improve CRISPR applications for enhancing human cognitive capacity including raising conscious awareness, decreasing inattention, reducing mind-wandering, lowering craving, sharpening mental focus, increasing concentration, enhancing mindfulness, improving meditation, increasing intuition and improving ESP.

It is another object of the invention to enable CRISPR-assisted psychotherapy applications to address psychological and psychosomatic issues.

It is another object of the invention to provide CRISPR-assisted cognitive therapy applications for alleviating unwanted behaviors and fostering desired behaviors.

It is a final object of the invention to provide a system which is applicable to any form of CRISPR for editing any neuron gene or RNA transcript.

One aspect of the invention provides genetic engineering neuron editing biologics for gene knock-out, gene silencing and gene knock-in.

Another aspect provides transcriptional engineering neuron editing biologics for RNA knock-out, RNA silencing, RNA knock-in, RNA translational interference and micro-RNA suppression.

A further aspect provides brain region activators comprising individually or in combination transcranial pulsed ultrasound, transcranial magnetic stimulation, neurofeedback, perceptual isolation, virtual reality and psychotherapy.

### DESCRIPTION OF THE DRAWINGS

FIG. 1 is an illustration of examples of neuron editing biologics.

FIG. 2 is an illustration of examples of brain region activation techniques.

FIGS. 3A and 3B illustrate a method for treating neurological conditions.

FIGS. 4A and 4B illustrate a method for cognitive enhancement.

### DETAILED DESCRIPTION

### I. Definitions

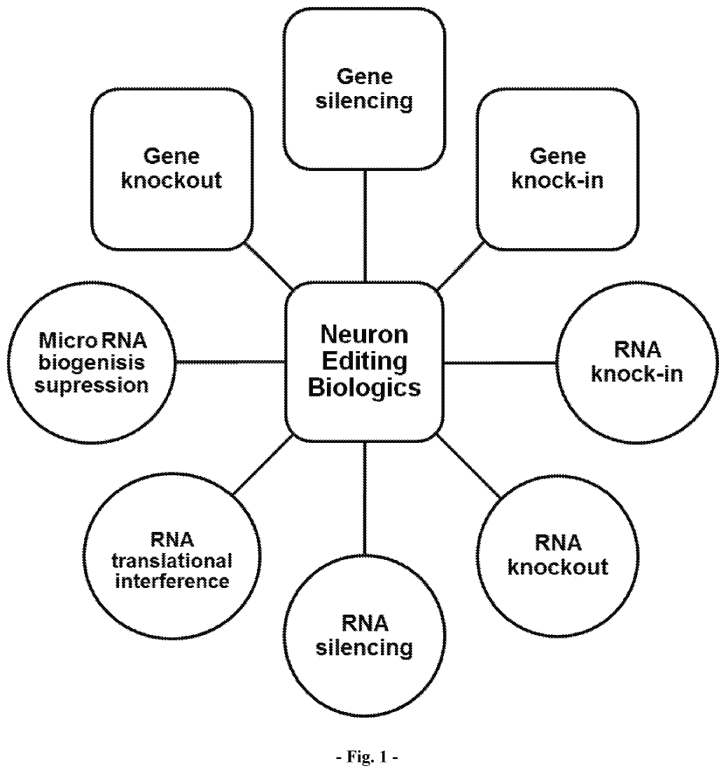

1. Neuron editing biologics are protein-based biopharmaceuticals which can modify a neuron's DNA or RNA. FIG. 1 illustrates 8 examples of neuron-editing biologics which are explained below. Other neuron editing biologics can be devised by those skilled in the art.

a) Genetic engineering neuron editing biologics
1. Gene knockout: The biologic is a catalytically-active gene-editing endonuclease complexed with a synthetic guide RNA.
2. Gene silencing: The biologic is a catalytically-inactive gene-editing endonuclease complexed with a synthetic guide RNA.
3. Gene knock-in: The biologic is a gene-editing endonuclease complexed with a gene-expression inhibiting nucleotide and a synthetic guide RNA.

b) Transcriptional engineering neuron editing biologics
1. RNA knockout: The biologic is a catalytically-active RNA-editing ribonuclease complexed with a single guide RNA to alter RNA nucleotides to repress gene translation.
2. RNA silencing: The biologic is a catalytically-inactive RNA-editing ribonuclease complexed with a single guide RNA to bind to RNA nucleotides to repress gene translation.
3. RNA knock-in: The biologic is a catalytically-inactive RNA-editing ribonuclease complexed with a single guide RNA and a deaminase enzyme to cause RNA nucleobase substitutions which result in translational interference.
4. RNA translational interference: The biologic is an RNA-editing ribonuclease complexed with an RNA-expression inhibiting nucleotide and a single guide RNA to alter RNA nucleotides to cause translational interference.
5. Micro-RNA biogenesis suppression: The biologic is a catalytically-active ribonuclease complexed with a single guide RNA to alter nucleotides in biogenesis processing sites for micro-RNA used in the translation of genes in order to reduce their expression.

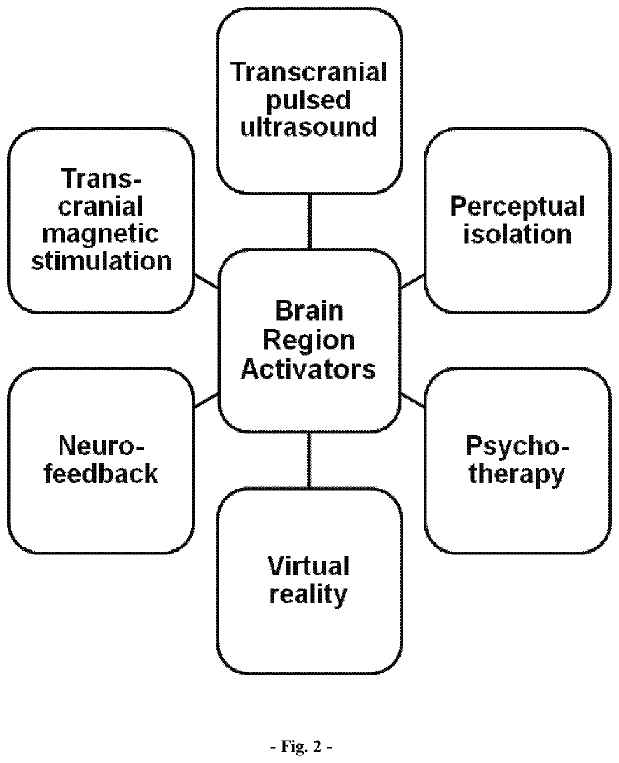

2. Brain region activators are methods and techniques for focusing and concentrating the neuronal activity in a subject's brain into a specific, targeted area of the brain. FIG. 2 illustrates 6 examples of brain region activators which are explained below. Other brain region activation methods can be devised by those skilled in the art.
1. Transcranial pulsed ultrasound is a technique which uses low-power, low-frequency ultrasound to stimulate high neuron activity in the brain. It can be directed to any brain region and precisely focused to areas as small as several cubic millimeters.
2. Transcranial magnetic stimulation is a form of neurostimulation which uses a shallow magnetic field to induce electric current to flow in small targeted regions near the surface of the brain.
3. Perceptual isolation is the deliberate removal of stimuli from the senses. Examples of perceptual isolation methods include sleep masks, white noise, soundproofing, or even floatation tanks.
4. Neurofeedback is a type of biofeedback that measures brain waves to produce a signal that can be used as feedback to teach self-regulation of brain function. Subjects alter their brain activity to increase performance on certain tasks, which changes the signal and increases cerebral blood flow to a specified region of the brain.
5. Virtual reality is an immersive, interactive, computer-generated experience which occurs in a simulated environment including auditory and visual feedback.
6. Psychotherapy is a wide field encompassing the use of hundreds of different methods and techniques to improve an individual's well-being, behavior and mental health, including cognitive therapy and other forms of therapies.

### II. Overview

### 1. Introduction

There is currently no known way to deliver neuron-editing biologics to targeted regions in the brain in order to achieve specific neurological treatment or cognitive enhancement goals. Although CRISPR can distinguish neurons from other types of cells in the body, it cannot differentiate between various areas in the brain.

This application describes methods and techniques for directing the delivery of neuron-editing biologics to targeted regions of the brain. These methods and techniques will enable the development of a rich new set of neurological treatment and cognitive enhancement applications.

### 2. Hemodynamics

Glucose and oxygen are the fuel which powers neuronal activity. The more active a neuron is, the faster it burns glucose and oxygen. Unlike other types of cells, neurons cannot store glucose, so their supply must be immediately replenished as soon as it is consumed. A circulatory system principle known as hemodynamics ensures the vital nutrients of glucose and oxygen are rapidly delivered to active neurons. Hemodynamics is well understood in the art, and tracking cerebral blood flow to measure neural activity is the basis for functional magnetic resonance neuroimaging (MU), and single photon emission computed tomography (SPECT); two of the most widely-used brain imaging techniques used in modern neuroscience.

### 3. Hemodynamic Vectoring

The methods of directing CRISPR molecules to specific types of cells are known as vectoring. Current CRISPR vectoring technology can target neurons, but it cannot provide navigational specificity to individual areas within the brain. However, CRISPR biologics in a subject's bloodstream can be directed along with glucose and oxygen to specific active areas in the brain by the principles of hemodynamics. Hemodynamics can draw CRISPR into active neurons which are firing.

By employing a selective brain region activation process to concentrate the subjects neural activity into a targeted brain region, we can use hemodynamics to draw cerebral blood flow containing CRISPR neuron editing biologics into the region.

Many kinds of brain region activation processes can be designed by cognitive neuroscience professionals to serve different applications and objectives. Examples of 6 brain region activation processes are provided hereinbelow. The invention gives cognitive neuroscience professionals a framework and platform to design, develop and deploy a variety of applications.

### III. Methodology

A. Treatment

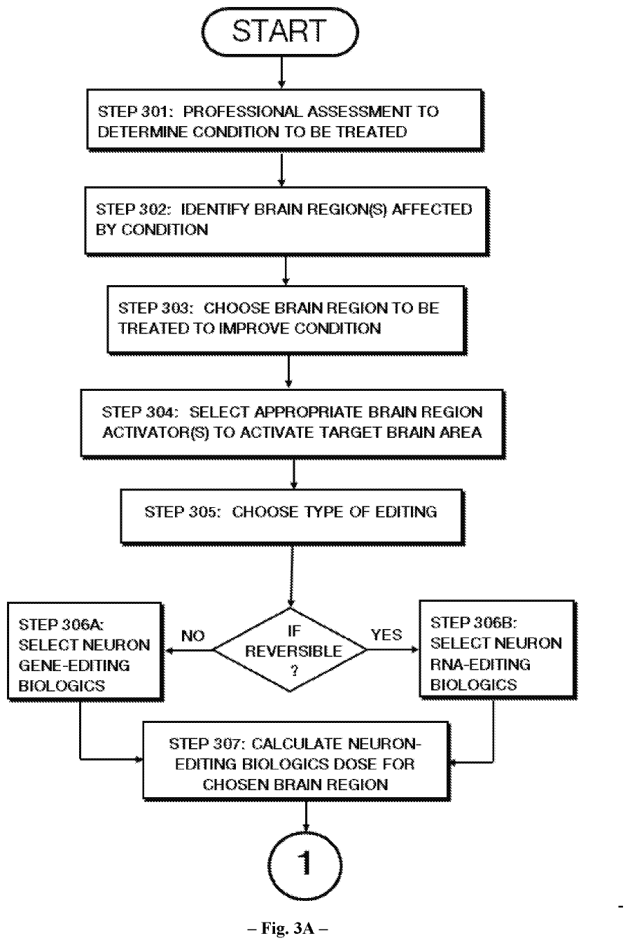

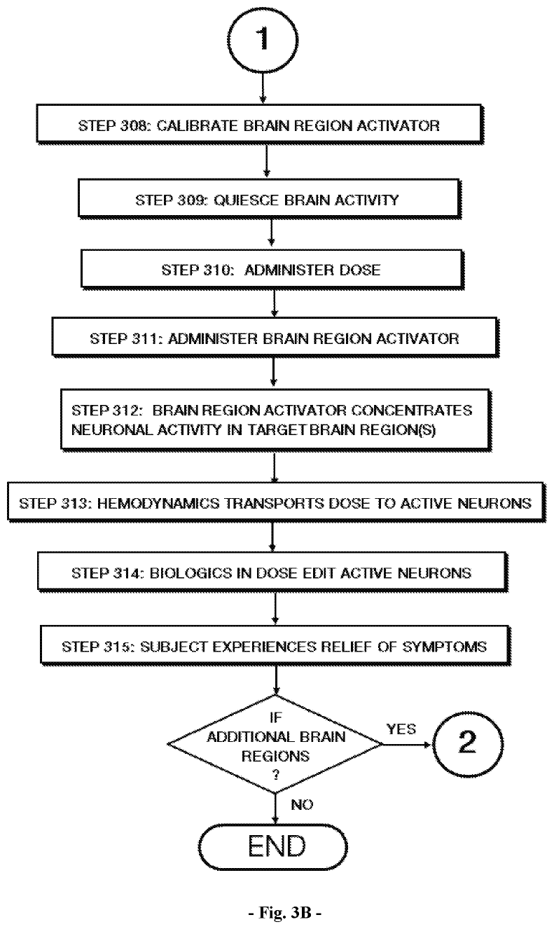

FIGS. 3A and 3B illustrate a method for using neuron editing biologics in combination with brain region activators to treat neurological conditions and disorders. Referring to FIG. 3A:

Step 301: Professional Assessment to Determine Condition to be Treated

A professional neuroscience practitioner conducts an assessment of the subject to identify the specific neurological issues, conditions or disorders to be treated. The practitioner can be a human or an AI computer program.

Step 302: Identify Brain Region(S) Affected by Condition

Neurological conditions and disorder may cause under- or over-activation in multiple areas in the brain. The practitioner will order a neuroimaging study, such as an fMRI or SPECT brain scan, to pinpoint area(s) in the subject's brain which are over- or under-activated. The brain scan will typically show abnormal activity in one or more regions, depending on the condition to be treated. Brain regions typically affected by 12 common neurological conditions are shown in Table 1.

Step 303: Choose Brain Region to be Treated to Improve Condition

The practitioner will establish treatment priorities according to the degree of over- or under-activation of each region as revealed in the neuroimaging study, beginning with the most over- or under-activated area.

Step 304: Select Appropriate Brain Region Activator(S) to Activate Target Brain Area

Table 1 illustrates 6 examples of brain region activators suitable for 12 neurological treatment applications. Other brain region activation methods can be devised by those skilled in the art. The activator examples shown are:

a) Transcranial pulsed ultrasound (TPU) can be effective in a variety of applications because it can be focused in almost any area of the brain with a precision down to a few square millimeters. Furthermore, TPU also increases the permeability of the brain's blood brain barrier, more effectively transporting neuron-editing biologics in the bloodstream to the brain areas it targets. TPU could also be used in combination with heat-sensitive CRISPR vectors, such as lipid nanoparticles, to provide very precise delivery targeting.

b) Transcranial magnetic stimulation (TMS) has been used successfully in the treatment of depression. Its effective range is limited to neurons near the brain's surface.

c) Neurofeedback has been shown to be effective across a wide spectrum of applications. It can activate almost any area in the brain.

d) Perceptual isolation is used in applications addressing the default mode network (DMN) (posterior cingulate cortex, medial prefrontal cortex, angular gyrus) to quiesce neural activity in other areas of the brain and concentrate neural activity into the DMN.

e) Virtual reality's primary use is in applications treating aversive memories. A VR simulation can strongly re-activate the memory. Genetic or transcriptional therapies can attenuate neuronal activity associated with the unwanted memories.

f) Psychotherapy can have efficacy in applications treating aversive memories, stress, anxiety and depression. By reactivating unwanted memories or conditions, psychotherapy can induce genetic or transcriptional therapies to attenuate their associated neuronal activity.

Step 305: Choose Type of Editing

Transcriptional editing is used to produce a reversible, temporary result for testing purposes. Genetic editing is selected when the subject is ready for a permanent treatment.

Step 306: Select Neuron-Editing Biologic S

The brain region selected for treatment may be over- or under-active. If the region is underactive, the practitioner selects neuron-editing biologics which raise neuron excitability to increase activity in the region. If it is overactive, the practitioner selects neuron-editing biologics which reduce neuron excitability in order to calm the region down. For example, a neuron's excitability can be reduced by decreasing its receptor population, which raises its electrical resistance. One way to achieve this is to edit gene HTR2A or its transcriptional or translational pathways to reduce its expression, which will lower the population of serotonin 2A receptors, as described in co-pending application Ser. No. 15/970,037.

Step 307: Calculate Neuron-Editing Biologics Dose for Chosen Brain Region.

Neuron editing biologics dosing is well known in the art. An example of a dosing formula appears in co-pending application Ser. No. 15/970,037.

Referring to FIG. 3B:

Step 308: Calibrate Brain Region Activator

The practitioner calibrates the brain region activator method chosen in Step 304 to address the subject's target brain region. For transcranial pulsed ultrasound or transcranial magnetic stimulation, the practitioner focuses the device on the target area in the subject's brain. For neurofeedback, the practitioner selects a neurofeedback program designed to address the target brain region. For perceptual isolation, the practitioner selects the stimuli reduction method to be used. For virtual reality, the practitioner chooses a VR simulation designed to evoke a particular memory or emotion related to the subject's issue. For psychotherapy, the practitioner selects a session protocol which evokes the subject's unwanted memory or condition.

Step 309: Quiesce Brain Activity

The subject is placed in a resting state free from distractions to minimize brain activity. This will tend to deactivate all brain regions except the default mode network.

Step 310: Administer Dose

Neuron-editing biologics doses can be administered to subjects via sublingual, oral, or transdermal application or through other methods well known in the art.

Step 311: Administer Brain Region Activator

The practitioner administers the brain region activator method chosen for the subject in Step 304.

Step 312: Brain Region Activator Concentrates Neuronal Activity in Target Brain Region(S).

The brain region activator concentrates neuronal activity in the target brain region(s).

Step 313: Hemodynamics Transports Dose to Active Neurons

Cerebral blood flow containing glucose, oxygen and neuron editing biologics is transported to active neurons in the targeted area of the subject's brain.

Step 314: Biologics in Dose Edit Active Neurons

Neuron editing biologics transfect neurons in the target area of the subject's brain and edit the neurons' DNA or RNA to normalize activity in the region.

Step 315: Subject Experiences Relief of Symptoms

Normalized brain region activity relieves the symptoms of the subject's neurological condition.

Step 316: Repeat Process for Additional Brain Regions as Needed

If multiple brain areas are indicated for treatment, the practitioner returns to Step 302 to address the next area of priority. The brain scan performed at this juncture verifies normalized brain activity in the region just treated.

Table 1 illustrates the practical application of the method just described in treating 12 examples of common neurological conditions. Referring specifically to Table 1:

1. ADD/ADHD—Attention Deficit Disorder (ADD) and Attention Deficit Hyperactivity Disorder (ADHD) may involve underactivation of the amygdala, ventromedial prefrontal cortex and hippocampus, depending on the ADD or ADHD subtype. Activity in these areas can be bolstered by administering neuron-editing biologics designed to increase neuron excitability, in conjunction with transcranial pulsed ultrasound or neurofeedback brain region activation techniques which direct the biologics to the amygdala, ventromedial prefrontal cortex and/or hippocampus via hemodynamic vectoring.

ADD and ADHD may also involve overactivation of the dorsal attention and default mode networks. Activity in these areas can be reduced by administering neuron-editing biologics designed to lower neuron excitability, in conjunction with transcranial pulsed ultrasound, neurofeedback and/or perceptual isolation brain region activation techniques which direct the biologics to the dorsal attention and/or default mode networks via hemodynamic vectoring.

2. Traumatic memory/PTSD—Post Traumatic Stress Disorder (PTSD) may involve overactivation of the amygdala and hippocampus. These areas can be calmed down by administering neuron-editing biologics for lowering neuron excitability, in conjunction with transcranial pulsed ultrasound, neurofeedback, virtual reality or psychotherapy brain region activation techniques which direct the biologics to the amygdala and/or hippocampus via hemodynamic vectoring.

PTSD also may cause underactivation of the ventromedial prefrontal cortex. Activity in this area can be stimulated by neuron-editing biologics for increasing neuron excitability, in conjunction with transcranial pulsed ultrasound, neurofeedback, virtual reality or psychotherapy brain region activation techniques which direct the biologics to the ventromedial prefrontal cortex via hemodynamic vectoring.

TABLE 1
Treatment

| | | Brain region activator | | | | | | |
|---|---|---|---|---|---|---|---|---|
| | Brain regions | Change | Transcranial | Transcranial | | Perceptual | Virtual | |
| Issue | affected | activity | ultrasound | magnetic stim. | Neurofeedback | isolation | reality | Psychotherapy |
| 1. ADD/ADHD | Amygdala, ventromedial | ↑ | • | | • | | | |
| | prefrontal cortex, | | | | | | | |
| | hippocampus | | | | | | | |
| | (under-activated) | | | | | | | |
| | Dorsal attention and | ↓ | • | | • | • | | |
| | default mode | | | | | | | |
| | networks (over-activated) | | | | | | | |
| 2. Traumatic | Amygdala, hippocampus | ↓ | • | | • | | • | • |
| memory/PTSD | Ventromedial prefrontal | ↑ | • | | • | | • | • |
| | cortex | | | | | | | |
| 3. OCD | Orbital gyrus, caudate | ↓ | • | | • | | | |
| | nucleus, dorsal anterior | | | | | | | |
| | cingulate cortex | | | | | | | |
| 4. Stress | Amygdala, hypothalamus | ↓ | • | | • | | | • |
| disorders | | | | | | | | |
| 5. Anxiety | Amygdala | ↓ | • | | • | | | • |
| 6. Depression | Posterior cingulate | ↑ | • | • | • | • | | |
| | cortex, prefrontal | | | | | | | |
| | cortex (underactive) | | | | | | | |
| | Amygdala, hippocampus, | ↓ | • | | • | | | |
| | anterior cingulate | | | | | | | |
| | cortex (overactive) | | | | | | | |
| 7. Sleep | Prefrontal and parietal | ↓ | • | | • | • | | |
| issues | cortex, precuneus, | | | | | | | |
| | anterior cingulate, | | | | | | | |
| | mesial temporal, | | | | | | | |
| | thalamus and hypo- | | | | | | | |
| | thalamic arousal | | | | | | | |
| | centers, default-mode | | | | | | | |
| | network | | | | | | | |
| 8. Memory | Hippocampus, amygdala | ↑ | • | | • | | | |
| concerns | | | | | | | | |
| 9. Concussions | Frontal and temporal | ↑ | • | | • | | | |
| | lobes | | | | | | | |
| 10. Tourette | Basal ganglia | ↓ | • | | | | | |
| Syndrome | | | | | | | | |
| 11. Psychosomatic | Amygdala, hippocampus | ↓ | • | | • | | | • |
| issues | Ventromedial prefrontal | ↑ | • | | • | | | • |
| | cortex | | | | | | | |
| 12. Tinnitus | Auditory cortex | ↓ | • | | | | | |

3. OCD—Neuroimaging studies of people with Obsessive Compulsive Disorder (OCD) have revealed hyperactivity in the dorsal anterior cingulate cortex, orbital gyms and caudate nucleus. Activity in these areas can be normalized by administering neuron-editing biologics for reducing neuron excitability, together with transcranial pulsed ultrasound or neurofeedback brain region activation techniques which direct the biologics to the dorsal anterior cingulate cortex, orbital gyms and/or caudate nucleus via hemodynamic vectoring.

4. Stress disorders—Individuals with stress disorders exhibit hyperactivity in the amygdala and hypothalamus. These areas can be brought into balance by administering neuron-editing biologics for reducing neuron excitability, combined with transcranial pulsed ultrasound, neurofeedback or psychotherapy brain region activation techniques which direct the biologics to the amygdala and/or hypothalamus via hemodynamic vectoring.

5. Anxiety—Individuals experiencing anxiety exhibit hyperactivity in the amygdala, which can be normalized by administering neuron-editing biologics for reducing neuron excitability, combined with transcranial pulsed ultrasound, neurofeedback or psychotherapy brain region activation techniques which direct the biologics to the amygdala via hemodynamic vectoring.

6. Depression—Depression involves underactivation of the posterior cingulate cortex and prefrontal cortex. Activity in these areas can be bolstered by administering neuron-editing biologics designed to increase neuron excitability, in conjunction with transcranial pulsed ultrasound, transcranial magnetic stimulation, neurofeedback and/or perceptual isolation brain region activation techniques which direct the biologics to the posterior cingulate cortex and/or prefrontal cortex via hemodynamic vectoring.

Depression is also associated with overactivation of the amygdala, hippocampus and anterior cingulate cortex. Activity in these areas can be reduced by administering neuron-editing biologics designed to lower neuron excitability, in conjunction with transcranial pulsed ultrasound or neurofeedback brain region activation techniques which direct the biologics to the amygdala, hippocampus and/or anterior cingulate cortex via hemodynamic vectoring.

7. Sleep issues—Insomnia and other sleep issues can involve overactivation of the prefrontal and parietal cortex, precuneus, anterior cingulate, mesial temporal, thalamus and hypothalamic arousal centers, and the default-mode network. Activity in these areas can be normalized by administering neuron-editing biologics for reducing neuron excitability, in conjunction with transcranial pulsed ultrasound, neurofeedback and/or perceptual isolation brain region activation techniques which direct the biologics to the prefrontal and parietal cortex, precuneus, anterior cingulate, mesial temporal, thalamus and hypothalamic arousal centers, and/or the default-mode network via hemodynamic vectoring.

8. Memory concerns—Neuroimaging studies of individuals with memory problems reveal subnormal activity in the hippocampus and amygdala. These areas can be stimulated by administering neuron-editing biologics for increasing neuron excitability, in conjunction with transcranial pulsed ultrasound or neurofeedback brain region activation techniques which direct the biologics to the hippocampus and/or amygdala via hemodynamic vectoring.

9. Concussions—Head injuries may lower neuronal activity in the frontal and temporal lobes. Increased activity in these areas can be promoted by administering neuron-editing biologics for increasing neuron excitability, together with transcranial pulsed ultrasound or neurofeedback brain region activation techniques which direct the biologics to the frontal and/or temporal lobes via hemodynamic vectoring.

10. Tourette Syndrome—Studies of individuals with Tourette Syndrome show subnormal activity in the basal ganglia. Activity can be normalized by administering neuron-editing biologics for increasing neuron excitability, along with transcranial pulsed ultrasound brain region activation techniques which direct the biologics to the basal ganglia via hemodynamic vectoring.

11. Psychosomatic issues—Psychosomatic issues may involve overactivation of the amygdala and hippocampus. These areas can be calmed down by administering neuron-editing biologics for lowering neuron excitability, in conjunction with transcranial pulsed ultrasound, neurofeedback, or psychotherapy brain region activation techniques which direct the biologics to the amygdala and/or hippocampus via hemodynamic vectoring.

Psychosomatic issues may also cause underactivation of the ventromedial prefrontal cortex. Activity in this area can be stimulated by neuron-editing biologics for increasing neuron excitability, in conjunction with transcranial pulsed ultrasound, neurofeedback, or psychotherapy brain region activation techniques which direct the biologics to the ventromedial prefrontal cortex via hemodynamic vectoring.

12. Tinnitus—Persistent ringing in the ears is caused by an overactivated auditory cortex. Activity can be reduced by administering neuron-editing biologics for decreasing neuron excitability, along with transcranial pulsed ultrasound brain region activation techniques which direct the biologics to the auditory cortex via hemodynamic vectoring.

B. Enhancement

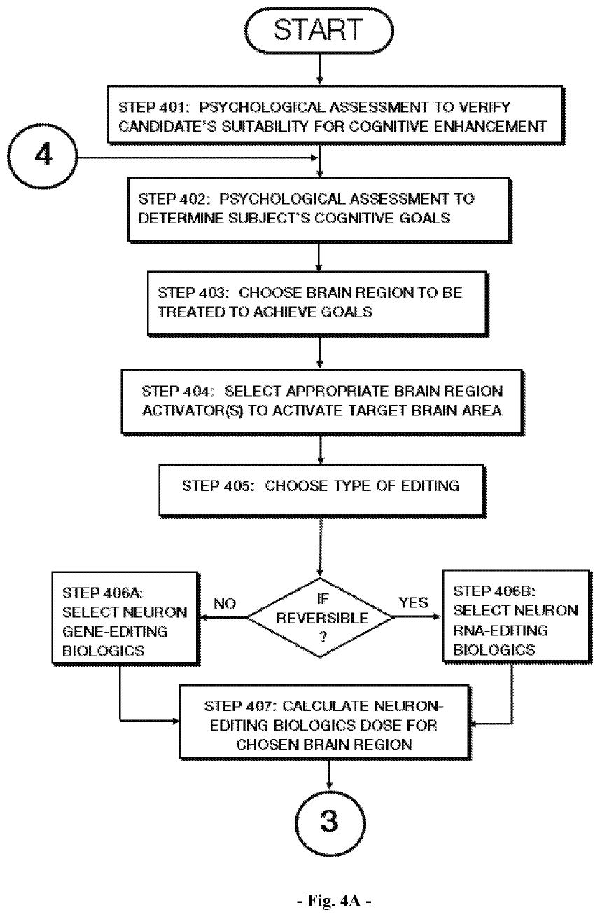

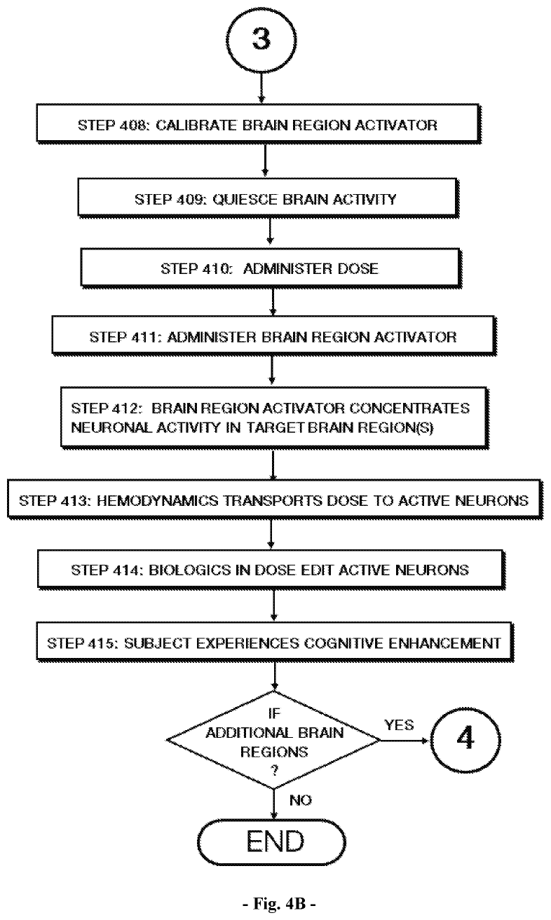

FIGS. 4A and 4B illustrate a method for using neuron editing biologics in combination with brain region activators to enhance cognitive abilities. Referring to FIG. 4A:

Step 401: Psychological Assessment to Verify Candidate's Suitability for Cognitive Enhancement

General-purpose genetic cognitive enhancement is suitable for adults in sound mental and emotional health. The process begins with a psychological assessment to screen out candidates who do not meet this criteria, for example, individuals with alcohol or substance abuse, bipolar disorder, depression, schizophrenia or other psychological conditions or disorders.

The assessment also ensures the candidate is not currently taking any drugs, medications or substances that could interfere with the normal, natural functioning of their brain; for example, certain prescription drugs, alcohol, caffeine, nicotine, cannabis, nootropics, ginseng or other similar substances or herbal preparations.

Candidates who satisfactorily meet the psychological assessment criteria are accepted as subjects for cognitive enhancement.

Step 402: Psychological Assessment to Determine Subject's Cognitive Goals

The second step is a psychological assessment to ascertain the subject's cognitive enhancement goals. This assessment covers several topics, including the type of cognitive enhancement the subject desires and whether the cognitive upgrade is to be permanent or temporary.

Step 403: Choose Brain Region to be Treated to Achi Ve Goals

Target brain regions for 13 cognitive enhancement applications are illustrated in Table 2. Genetic or transcriptional engineering can optimize neuron performance in these regions in order to achieve the subject's goals. Depending on the application, optimization may involve increasing or decreasing neuronal activity.

Step 404: Select Appropriate Brain Region Activators(s) to Activate Target Brain Area

Table 2 illustrates 6 examples of brain region activators suitable for 13 cognitive enhancement applications. Other brain region activation methods can be devised by those skilled in the art. The activator examples shown are identical to the ones described in Step 404.

Step 405: Choose Type of Editing

Transcriptional editing is used to produce a reversible, temporary result for testing purposes. Genetic editing is selected when the subject is ready for a permanent cognitive enhancement.

Step 406: Select Neuron-Editing Biologics

The brain region selected for enhancement may be over- or under-active. If the region is underactive, the practitioner selects neuron-editing biologics which raise neuron excitability to increase activity in the region. If it is overactive, the practitioner selects neuron-editing biologics which reduce neuron excitability in order to calm the region down. For example, a neuron's excitability can be reduced by decreasing its receptor population, which raises its electrical resistance. One way to achieve this is to edit gene HTR2A or its transcriptional or translational pathways to reduce its expression, which will lower the population of serotonin 2A receptors, as described in co-pending application Ser. No. 15/970,037.

Step 407: Calculate Neuron-Editing Biologics Dose for Chosen Brain Region

Neuron editing biologics dosing is well known in the art. An example of a dosing foi mula appears in co-pending application Ser. No. 15/970,037.

Referring to FIG. 4B:

Step 408: Calibrate Brain Region Activator

The practitioner calibrates the brain region activator method chosen in Step 304 to address the subject's target brain region. For transcranial pulsed ultrasound, the practitioner focuses the device on the target area in the subject's brain. For perceptual isolation, the practitioner selects the stimuli reduction method to be used. For cognitive therapy, the practitioner selects a psychotherapy session protocol which evokes either the subject's unwanted behaviors or their desired behaviors.

Step 409: Quiesce Brain Activity

The subject is placed in a resting state free from distractions to minimize brain activity: This will tend to deactivate all brain regions except the default mode network.

Step 410: Administer Dose

Neuron-editing biologics doses can be administered to subjects via sublingual, oral, or transdermal application or through other methods well known in the art.

Step 411: Administer Brain Region Activator

The practitioner administers the brain region activator method chosen for the subject in Step 404.

Step 412: Brain Region Activator Concentrates Neuronal Activity in Target Brain Region(S)

The brain region activator concentrates neuronal activity in the target brain region(s).

Step 413: Hemodynamics Transports Dose to Active Neurons

Cerebral blood flow containing glucose, oxygen and neuron editing biologics is transported to active neurons in the targeted area of the subject's brain.

Step 414: Biologics in Dose Edit Active Neurons

Neuron editing biologics transfect neurons in the target area of the subject's brain and edit the neurons' DNA or RNA to optimize activity in the region.

Step 415: Subject Experiences Cognitive Enhancement

Optimized brain region activity expands the subject's cognitive capacity.

Step 416: Repeat Process for Additional Brain Regions as Needed

If multiple brain areas are indicated for cognitive enhancement, the practitioner returns to Step 402 to address the next area of priority. The brain scan performed at this juncture verifies optimized brain activity in the region just treated.

Table 2 illustrates the practical application of the method just described in producing 13 distinct types of cognitive enhancements. Referring specifically to Table 2:

1-9 General-purpose cognitive enhancement—Table 2 illustrates a method for achieving 9 different types of general-purpose cognitive enhancements, including raising conscious awareness, decreasing inattention, reducing mind wandering, lowering craving, sharpening mental focus, increasing concentration, enhancing mindfulness, improving meditation and increasing intuition. These objectives can be achieved by administering neuron-editing biologics for decreasing neuron excitability, along with transcranial pulsed ultrasound and/or perceptual isolation brain region activation techniques which direct the biologics to the ventral posterior cingulate cortex (PCC) via hemodynamic vectoring. Reduced PCC activity is experimentally correlated with increased attention, conscious awareness, mental acuity, clarity, focus, concentration, and mindfulness, and with reduced mind-wandering, inattention and cravings.

10. Improve ESP—Studies have shown individuals who can demonstrate extra-sensory perception (ESP) abilities exhibit higher activity in the brain's caudate region. Subjects who desire to raise their ESP abilities may benefit from administering neuron-editing biologics for increasing neuron excitability, along with transcranial pulsed ultrasound brain region activation techniques which direct the biologics to the caudate via hemodynamic vectoring.

11. Cognitive therapy for unwanted behaviors—Psychotherapy session protocols can be designed to stimulate the underlying causes of the subject's unwanted behavior, including attitudes, emotions, beliefs, expectations and memories. This mental activity will generate brainwave and neural activity in corresponding areas in the subject's brain. Neuron editing biologics administered for reducing neuron excitability will be transported to these active areas via hemodynamic vectoring. Actively-firing neurons along the neural pathways traveled by brainwaves associated with the issue will absorb the edits. After treatment, when the issue is consciously or subconsciously stimulated, its associated brainwaves will travel through less-conductive neurons, reducing their power.

12. Cognitive therapy for desired behaviors—Psychotherapy session protocols and immersive multi-sensory VR programs can be designed to help the subject visualize and experience desired behaviors, including attitudes, feelings, beliefs, expectations and assumptions. This mental activity will generate brainwave and neural activity in corresponding areas in the subject's brain. Neuron editing biologics administered for increasing neuron excitability will be transported to these active areas via hemodynamic vectoring. Actively-firing neurons along the neural pathways traveled by brainwaves associated with the desired behavior will absorb the edits. After treatment, when the behavior is initiated, its associated brainwaves will travel through more-conductive neurons, raising their power.

TABLE 2
Enhancement

| | | | | | | | | |
|---|---|---|---|---|---|---|---|---|
| | | Change | Transcranial | Transcranial | | Perceptual | Virtual | |
| Objective | Brain Regions | activity | ultrasound | magnetic stim. | Neurofeedback | isolation | reality | Psychotherapy |

| | | | | | | |
|---|---|---|---|---|---|---|
| 1. Raise conscious | Posterior | ↓ | • | • | | |
| awareness | cingulate | | | | | |
| 2. Decrease | cortex | | • | • | | |
| inattention | (ventral) | | | | | |
| 3. Reduce mind- | | | • | • | | |
| wandering | | | | | | |
| 4. Lower craving | | | • | • | | |
| 5. Sharpen mental | | | • | • | | |
| focus | | | | | | |
| 6. Increase | | | • | • | | |
| concentration | | | | | | |
| 7. Enhance | | | • | • | | |
| mindfulness | | | | | | |
| 8. Improve | | | • | • | | |
| meditation | | | | | | |
| 9. Increase | | | • | • | | |
| intuition | | | | | | |
| 10. Improve | Caudate | ↑ | • | | | |
| ESP | | | | | | |
| 11. Cognitive | Various | ↓ | | | | • |
| therapy for | | | | | | |
| unwanted behaviors | | | | | | |
| 12. Cognitive | Various | ↑ | | | • | • |
| therapy for | | | | | | |
| desired behaviors | | | | | | |
| 13. Alleviate | Posterior | ↓ | • | • | | |
| cognitive | cingulate | | | | | |
| impairment | cortex | | | | | |
| symptoms | | | | | | |

13. Alleviate cognitive impairment symptoms—Mild cognitive impairment (MCI) may affect many different regions of the brain. It can be treated indirectly by administering a cognitive enhancement protocol to offset its effects. This protocol involves administering neuron-editing biologics for decreasing neuron excitability in the posterior cingulate cortex (PCC), in conjunction with transcranial pulsed ultrasound or perceptual isolation techniques which direct the biologics to the PCC via hemodynamic vectoring.

Although specific embodiments of the invention have been disclosed herein in detail, it is to be understood that this is for the purpose of illustrating the invention, and should not be construed as necessarily limiting the scope of the invention, since it is apparent that many changes can be made to the disclosed methods by those skilled in the art to suit particular applications.

We claim:

1. A method for modifying neurons in targeted brain regions comprising administering a neuron-editing biologic in combination with a brain region activator which directs the neuron-editing biologic to a specific area in the brain via hemodynamics.

2. A method as recited in claim 1 where the neuron-editing biologic is a catalytically-active gene-editing endonuclease complexed with a synthetic guide RNA.

3. A method as recited in claim 1 where the neuron-editing biologic is a catalytically-inactive gene-editing endonuclease complexed with a synthetic guide RNA.

4. A method as recited in claim 1 where the neuron-editing biologic is a gene-editing endonuclease complexed with a gene-expression inhibiting nucleotide and a synthetic guide RNA.

5. A method as recited in claim 1 where the neuron-editing biologic is a catalytically-active RNA-editing ribonuclease complexed with a single guide RNA to alter RNA nucleotides to repress gene translation.

6. A method as recited in claim 1 where the neuron-editing biologic is a catalytically-inactive RNA-editing ribonuclease complexed with a single guide RNA to bind to RNA nucleotides to repress gene translation.

7. A method as recited in claim 1 where the neuron-editing biologic is a catalytically-inactive RNA-editing ribonuclease complexed with a single guide RNA and a deaminase enzyme to cause RNA nucleobase substitutions which result in translational interference.

8. A method as recited in claim 1 where the neuron-editing biologic is an RNA-editing ribonuclease complexed with an RNA-expression inhibiting nucleotide and a single guide RNA to alter RNA nucleotides to cause translational interference.

9. A method as recited in claim 1 where the neuron-editing biologic is a catalytically-active ribonuclease complexed with a single guide RNA to alter nucleotides in biogenesis processing sites for micro-RNA used in the translation of genes in order to reduce their expression.

10. A method as recited in claim 1 where the brain region activator is transcranial pulsed ultrasound.

11. A method as recited in claim 1 where the brain region activator is transcranial magnetic stimulation.

12. A method as recited in claim 1 where the brain region activator is neurofeedback.

13. A method as recited in claim 1 where the brain region activator is perceptual isolation.

14. A method as recited in claim 1 where the brain region activator is a virtual reality program.

15. A method as recited in claim 1 where the brain region activator is psychotherapy.
